(19) United States
(12) Patent Application Publication
(10) Pub. No.: US 2014/0276023 A1
(43) Pub. Date: Sep. 18, 2014

(54) SYSTEMS AND METHODS FOR DETERMINING A PROBABILITY OF A FEMALE SUBJECT HAVING A CARDIAC EVENT

(71) Applicant: VOLCANO CORPORATION, San Diego, CA (US)

(72) Inventors: Donna Collins, San Diego, CA (US); Mary Lynn Gaddis, San Diego, CA (US); Nathaniel J. Kemp, Concord, MA (US); Bernhard Sturm, Davis, CA (US)

(73) Assignee: VOLCANO CORPORATION, San Diego, CA (US)

(21) Appl. No.: 14/205,683

(22) Filed: Mar. 12, 2014

Related U.S. Application Data
(60) Provisional application No. 61/777,839, filed on Mar. 12, 2013.

(51) Int. Cl. A61B 5/00; A61B 8/12

(52) U.S. Cl. CPC A61B 5/0035; A61B 8/12; A61B 5/0066
USPC 600/427; 600/443; 600/425

(57) ABSTRACT

The invention generally relates to systems and methods for determining a probability of a female subject having a cardiac event.

### RELATED APPLICATION

The present application claims the benefit of and priority to U.S. provisional patent application Ser. No. 61/777,839, filed Mar. 12, 2013, the content of which is incorporated by reference herein in its entirety.

### FIELD OF THE INVENTION

The invention generally relates to systems and methods for determining a probability of a female subject having a cardiac event.

### BACKGROUND

The identification of problematic cardiovascular lesions in men is relatively simple compared to women. In men, accumulation of plaque often leads to an acute stenosis than can easily be identified, for example via fractional flow reserve determination. In women, however, plaque accumulates diffusely throughout the vessel, rather than acutely in one spot. That diffuse distribution does not signal the necessity of therapeutic intervention in the same manner as acute accumulation. With the problem undetected, plaque will continue to accumulate. Often, women will test negative for serious coronary conditions using conventional methods only to suffer a major adverse cardiovascular event several months later when plaque has accumulated past a certain threshold.

### SUMMARY

The present invention overcomes this problem by using an algorithm to calculate a risk associated with a particular cardiac lesion. Generally, aspects of the invention are accomplished by using data from a cohort of women for whom image data and cardiac disease outcome are known. A female subject undergoes an imaging procedure that obtains image data representative of an inside of a vessel. That data is run through the algorithm that has been trained on the reference set. The algorithm then assigns a risk score to the subject based on comparing the subject's image data against the physical data in the reference set. In certain aspects, a risk score is assigned to a particular lesion.

In certain aspects, the invention provides systems for determining a probability of a female subject having a cardiac event. The system typically includes a central processing unit (CPU), and storage coupled to said CPU for storing instructions. The instructions, when executed by the CPU, cause the CPU to accept as input, data representative of an inside of a vessel of a female subject. The CPU is then causes to provide a probability of the subject having a cardiac event as a result of running an algorithm on the input data. The algorithm has been trained on a reference set of data from a plurality of women for whom image data and cardiac disease outcome are known.

In other aspects, the invention provides methods for determining a probability of a female subject having a cardiac event. Those methods involve accepting as input, data representative of an inside of a vessel of a female subject. Additionally, the methods involve providing a probability of the subject having a cardiac event as a result of running an algorithm on the input data. The algorithm has been trained on a reference set of data from a plurality of women for whom image data and cardiac disease outcome are known.

Any medical intervention that obtains data about an inside of a vessel may be used with systems and methods of the invention. Exemplary imaging techniques include Exemplary imaging devices include optical coherence tomography (OCT), spectroscopic devices, (including fluorescence, absorption, scattering, and Raman spectroscopies), intravascular ultrasound (IVUS), Forward-Looking IVUS (FLIVUS), high intensity focused ultrasound (HIFU), radiofrequency, optical light-based imaging, magnetic resonance, radiography, nuclear imaging, photoacoustic imaging, electrical impedance tomography, elastography, pressure sensing wires, intracardiac echocardiography (ICE), forward looking ICE and orthopedic, spinal imaging and neurological imaging, image guided therapeutic devices or therapeutic delivery devices, diagnostic delivery devices, and the like.

In certain embodiments, the data also includes data obtained from an external imaging device that images from outside of the subject. In such embodiments, methods of the invention then involve combining external imaging data and internal data to produce an image of the vessel. Methods for combining external and internal image data are described, for example in Huennekens et al. (U.S. patent application number 2013/0030295), the content of which is incorporated by reference herein its entirety.

In addition to image data, the reference data set may also include data regarding other characteristics that impact cardiovascular health. Exemplary characteristics include age, weight, level of physical activity, smoking, or a combination thereof.

### BRIEF DESCRIPTION OF THE DRAWINGS

FIG. 1 illustrates a system for performing methods of the invention.

FIG. 2 is a process chart depicting the procedural steps for determining the probability of a female having a cardiac event, according to certain embodiments.

### DETAILED DESCRIPTION

The invention generally relates to systems and methods for determining a probability of a female subject having a cardiac event. Certain aspects of the invention are especially amenable for implementation using a computer. In those embodiments, systems and methods of the invention encompass a central processing unit (CPU) and storage coupled to the CPU. The storage stores instructions that when executed by the CPU, cause the CPU to accept as input, data representative of an inside of a vessel of a female subject. The CPU is then causes to provide a probability of the subject having a cardiac event as a result of running an algorithm on the input data. The algorithm has been trained on a reference set of data from a plurality of women for whom image data and cardiac disease outcome are known.

Any medical intervention that obtains data about an inside of a vessel may be used with systems and methods of the invention. Exemplary imaging techniques include Exemplary imaging devices include optical coherence tomography (OCT), spectroscopic devices, (including fluorescence, absorption, scattering, and Raman spectroscopies), intravascular ultrasound (IVUS), Forward-Looking IVUS (FLIVUS), high intensity focused ultrasound (HIFU), radiofrequency, optical light-based imaging, magnetic resonance, radiography, nuclear imaging, photoacoustic imaging, electrical impedance tomography, elastography, pressure sensing wires, intracardiac echocardiography (ICE), forward looking ICE and orthopedic, spinal imaging and neurological imaging, image guided therapeutic devices or therapeutic delivery devices, diagnostic delivery devices, and the like.

In certain embodiments, the imaging device is an OCT device. OCT systems and methods are generally described in Castella et al. (U.S. Pat. No. 8,108,030), Milner et al. (U.S. Patent Application Publication No. 2011/0152771), Condit et al. (U.S. Patent Application Publication No. 2010/0220334), Castella et al. (U.S. Patent Application Publication No. 2009/0043191), Milner et al. (U.S. Patent Application Publication No. 2008/0291463), and Kemp, (U.S. Patent Application Publication No. 2008/0180683), the content of each of which is incorporated by reference in its entirety. Additional description of OCT systems and methods is described in Kemp (U.S. Pat. No. 8,049,900), Kemp (U.S. Pat. No. 7,929,148), Milner (U.S. Pat. No. 7,853,316), Feldman et al. (U.S. Pat. No. 7,711,413), Kemp et al., U.S. Patent Application Publication No. 2012/0224751), Milner et al. (U.S. Patent Application Publication No. 2012/0136259), Kemp et al., (U.S. Patent Application Publication No. 2012/0013914), Milner et al. (U.S. Patent Application Publication No. 2011/0152771), and Kemp et al. (U.S. Patent Application Publication No. 2009/0046295), the content of each of which is incorporated by reference in its entirety.

OCT systems of the invention include a light source. The light source may be any light source generally used with OCT. Exemplary light sources include a narrow line width tunable laser source or a superluminescent diode source. Examples of narrow line width tunable laser sources include, but are not limited to, lasers having a Bragg diffraction grating or a deformable membrane, lasers having a spectral dispersion component (e.g., a prism), or Fabry-Pérot based tuning laser.

OCT systems of the invention also include an interferometer. The interferometer may be any interferometer generally used with OCT. Typically, the interferometer will have a differential beam path for the light or a common beam path for the light. In either case, the interferometer is operably coupled to the light source. In a differential beam path layout, light from a broad band light source or tunable laser source is input into an interferometer with a portion of light directed to a sample and the other portion directed to a reference surface. A distal end of an optical fiber is interfaced with a catheter for interrogation of the target tissue during a catheterization procedure. The reflected light from the tissue is recombined with the signal from the reference surface forming interference fringes (measured by a photovoltaic detector) allowing precise depth-resolved imaging of the target tissue on a micron scale. Exemplary differential beam path interferometers are Mach-Zehnder interferometers and Michelson interferometers. Differential beam path interferometers are further described for example in Feldman et al. (U.S. Pat. No. 7,783,337) and Tearney et al. (U.S. Pat. Nos. 6,134,003 and 6,421,164), the content of each of which is incorporated by reference herein in its entirety.

The differential beam path optical layout of the interferometer includes a sample arm and a reference arm. The sample arm is configured to accommodate and couple to a catheter. The differential beam path optical layout also includes optical circulators to. The circulators facilitate transmission of the emitted light in a particular direction. Circulators and their use in OCT systems are further described for example in B. Bouma et al. (Optics Letters, 24:531-533, 1999), the entire disclosure of which is incorporated herein by reference. In the interferometer, there is a circulator where the emitted light is split to the sample arm and the reference arm. The system also includes a circulator that directs light to the sample and receives reflected light from the sample and directs it toward a detector. The system also includes a circulator that directs light to the reference surface and received reflected light from the reference surface and directs it toward the detector. There is also a circulator at the point at which reflected light from the sample and reflected light from the reference are recombined and directed to the detector.

In a common beam path system, rather than splitting a portion of the light to a reference arm, all of the produced light travels through a single optical fiber. Within the single fiber is a reflecting surface. A portion of the light is reflected off that surface prior to reaching a target tissue (reference) and a remaining portion of the light passes through the reflecting surface and reaches the target tissue. The reflected light from the tissue recombines with the signal from the reference forming interference fringes allowing precise depth-resolved imaging of the target tissue on a micron scale. Common beam path interferometers are further described for example in Vakhtin, et al. (Applied Optics, 42(34):6953-6958, 2003), Wang et al. (U.S. Pat. No. 7,999,938), Tearney et al. (U.S. Pat. No. 7,995,210), and Galle et al. (U.S. Pat. No. 7,787,127), the content of each of which is incorporated by reference herein in its entirety.

The common beam path optical layout of the interferometer includes a single array of optical fibers that are connected to a circulator. The array of optical fibers are configured to accommodate and couple to a catheter. The circulator directs light transmitted from the light source through the array of optical fibers of the common beam path optical layout to a sample and reference, and receives the reflected light from the sample and reference and directs it to the detector.

OCT systems of the invention include a detector. The detector includes photodetection electronics. The detector can support both balanced and non-balanced detection. OCT detectors are described for example in Kemp (U.S. Pat. No. 8,049,900), Kemp (U.S. Pat. No. 7,929,148), Milner (U.S. Pat. No. 7,853,316), Feldman et al. (U.S. Pat. No. 7,711,413), Kemp et al., U.S. Patent Application Publication No. 2012/0224751), Milner et al. (U.S. Patent Application Publication No. 2012/0136259), Kemp et al., (U.S. Patent Application Publication No. 2012/0013914), Milner et al. (U.S. Patent Application Publication No. 2011/0152771), and Kemp et al. (U.S. Patent Application Publication No. 2009/0046295), the content of each of which is incorporated by reference in its entirety.

OCT systems of the invention may conduct any form of OCT known in the art. One manner for conducting OCT may be Swept-Source OCT (“SS-OCT”). SS-OCT time-encodes the wavenumber (or optical frequency) by rapidly tuning a narrowband light source over a broad optical bandwidth. The high speed tunable laser sources for SS-OCT exhibit a nonlinear or non-uniform wavenumber vs. time [k(t)] characteristic. As such, SS-OCT interferograms sampled uniformly in time [S(t), e.g., using an internal digitizer clock] must be remapped to S(k) before Fourier transforming into the path length (z) domain used to generate the OCT image. An SS-OCT system and methods for its use are described in Kemp et al., (U.S. Patent Application Publication No. 2012/0013914). The content of which is incorporated by reference herein in its entirety.

In other embodiments, the imaging device is an IVUS device. There are two types of IVUS catheters commonly in use, mechanical/rotational IVUS catheters and solid state catheters. A solid state catheter (or phased array) has no rotating parts, but instead includes an array of transducer elements (for example 64 elements). In a rotational IVUS catheter, a single transducer having a piezoelectric crystal is rapidly rotated (e.g., at approximately 1800 revolutions per minute) while the transducer is intermittently excited with an electrical pulse. The excitation pulse causes the transducer to vibrate, sending out a series of transmit pulses. The transmit pulses are sent at a frequency that allows time for receipt of echo signals. The sequence of transmit pulses interspersed with receipt signals provides the ultrasound data required to reconstruct a complete cross-sectional image of a vessel.

The general design and construction of IVUS catheters is shown, for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et al., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et al., U.S. Pat. No. 5,183,048, Eberle et al., U.S. Pat. No. 5,167,233, Eberle et al., U.S. Pat. No. 4,917,097, Eberle et al., U.S. Pat. No. 5,135,486, Corl U.S. Pat. App. No. 2010/0234736, Davies et al., U.S. Pat. No. 8,317,713, Stephens et al., U.S. Pat. No. 6,780,157, Nix et al., U.S. Pat. No. 7,037,269, and other references well known in the art relating to intraluminal ultrasound devices and modalities. The catheter will typically have proximal and distal regions, and will include an imaging tip located in the distal region. Such catheters have an ability to obtain echographic images of the area surrounding the imaging tip when located in a region of interest inside the body of a patient. The catheter, and its associated electronic circuitry, will also be capable of defining the position of the catheter axis with respect to each echographic data set obtained in the region of interest.

Besides intravascular ultrasound, other types of ultrasound catheters can be made using the teachings provided herein. By way of example and not limitation, other suitable types of catheters include non-intravascular intraluminal ultrasound catheters, intracardiac echo catheters, laparoscopic, and interstitial catheters. In addition, the probe may be used in any suitable anatomy, including, but not limited to, coronary, carotid, neuro, peripheral, or venous.

In certain embodiments, the data also includes data obtained from an external imaging device that images from outside of the subject. Exemplary external imaging technologies include angiography, MRI, CT, X-ray/angiography and ultrasound. In such embodiments, methods of the invention then involve combining external imaging data and internal data to produce an image of the vessel. Methods for combining external and internal image data are described, for example in Huennekens et al. (U.S. patent application number 2013/0030295), the content of which is incorporated by reference herein its entirety. An exemplary data package for image co-registration is sold by Sync-Rx (Netanya, Israel).

The information data from the female subject is then compared to a reference set of data in order to provide a probability of the female subject having a cardiac event. In certain aspects, the reference set includes data collected from of a cohort or plurality of women for whom image data and cardiac disease outcome are known. In addition to image data, other characteristics related to cardiovascular disease may also be obtained Such additional characteristics may include age, height, weight, smoking habits, alcohol intake, etc.

In some embodiments, methods and systems of the invention use a prognosis predictor for predicting risk of a cardiac event. The prognosis predictor can be based on any appropriate pattern recognition method that receives input data representative of an inside of a female's vessel and provides an output that indicates a probability of the female subject having a cardiac event. The prognosis predictor is trained with training data from a plurality of women for whom image data and cardiac disease outcome are known. The plurality of women used to train the prognosis predictor is also known as the training population. For each woman in the training population, the training data includes image data representative of an inside of a vessel. Various prognosis predictors that can be used in conjunction with the present invention are described below. In some embodiments, additional women having known image data and known disease outcomes can be used to test the accuracy of the prognosis predictor obtained using the training population. Such additional patients are known as the testing population.

In certain embodiments, the methods of invention use a prognosis predictor, also called a classifier, for determining the probability of the female subject having a cardiac event. As noted above, the prognosis predictor can be based on any appropriate pattern recognition method that receives a profile, such as a profile based on a plurality of images of an inside of a female's vessel and provides an output comprising data indicating a good prognosis or a poor prognosis, i.e., the risk of having a cardiac event or the risk of a specific lesion.

A prognosis predictor based on any of such methods can be constructed using the profiles and prognosis data of the training patients. Such a prognosis predictor can then be used to predict the risk of the female subject having a cardiac event based on her image data and potentially other factors.

In one embodiment, the prognosis predictor can be prepared by (a) generating a reference set of women for whom image data and cardiac outcome are known; (b) determining for each image, a metric of correlation between the image and cardiac outcome in a plurality of women having known cardiac outcomes outcomes at a predetermined time; (c) selecting one or more images based on the level of correlation; (d) training a prognosis predictor, in which the prognosis predictor receives image data representative of the images selected in the prior step and provides an output indicating a probability of the female subject having a cardiac event, with training data from the reference set of subjects including assessments of images taken from the women.

Various known statistical pattern recognition methods can be used in conjunction with the present invention. Suitable statistical methods include, without limitation, logic regression, ordinal logistic regression, linear or quadratic discriminant analysis, clustering, principal component analysis, nearest neighbor classifier analysis, and Cox proportional hazards regression. Non-limiting examples of implementing particular prognosis predictors in conjunction are provided herein to demonstrate the implementation of statistical methods in conjunction with the training set.

In some embodiments, the prognosis predictor is based on a regression model, preferably a logistic regression model. Such a regression model includes a coefficient for each of the images in a selected set of images. In such embodiments, the coefficients for the regression model are computed using, for example, a maximum likelihood approach.

Cox proportional hazards regression also includes a coefficient for each of the images in a selected set of images. Cox proportional hazards regression incorporates censored data (women in the reference set that did not return for treatment). In such embodiments, the coefficients for the regression model are computed using, for example, a maximum partial likelihood approach.

Some embodiments of the present invention provide generalizations of the logistic regression model that handle multicategory (polychotomous) responses. Such embodiments can be used to discriminate an organism into one or three or more prognosis groups. Such regression models use multicategory logit models that simultaneously refer to all pairs of categories, and describe the odds of response in one category instead of another. Once the model specifies logits for a certain (J-1) pairs of categories, the rest are redundant. See, for example, Agresti, An Introduction to Categorical Data Analysis, John Wiley & Sons, Inc., 1996, New York, Chapter 8, which is hereby incorporated by reference. Linear discriminant analysis (LDA) attempts to classify a subject into one of two categories based on certain object properties. In other words, LDA tests whether object attributes measured in an experiment predict categorization of the objects. LDA typically requires continuous independent variables and a dichotomous categorical dependent variable. In the present invention, the selected images serve as the requisite continuous independent variables. The prognosis group classification of each of the members of the training population serves as the dichotomous categorical dependent variable.

LDA seeks the linear combination of variables that maximizes the ratio of between-group variance and within-group variance by using the grouping information. Implicitly, the linear weights used by LDA depend on how selected images manifests in the two groups (e.g., a group that has a cardiac event and a group that does not) and how the selected image correlates with the manifestation of other images. For example, LDA can be applied to the data matrix of the N members in the training sample by K images. Then, the linear discriminant of each member of the training population is plotted. Ideally, those members of the training population representing a first subgroup (e.g. those subjects that have a major cardiac event) will cluster into one range of linear discriminant values and those member of the training population representing a second subgroup (e.g. those subjects that do not have a cardiac event) will cluster into a second range of linear discriminant values. The LDA is considered more successful when the separation between the clusters of discriminant values is larger. For more information on linear discriminant analysis, see Duda, Pattern Classification, Second Edition, 2001, John Wiley & Sons, Inc; and Hastie, 2001, The Elements of Statistical Learning, Springer, New York; Venables & Ripley, 1997, Modern Applied Statistics with s-plus, Springer, New York.

Quadratic discriminant analysis (QDA) takes the same input parameters and returns the same results as LDA. QDA uses quadratic equations, rather than linear equations, to produce results. LDA and QDA are interchangeable, and which to use is a matter of preference and/or availability of software to support the analysis. Logistic regression takes the same input parameters and returns the same results as LDA and QDA.

In some embodiments of the present invention, decision trees are used to classify patients using image data for a selected set of images. Decision tree algorithms belong to the class of supervised learning algorithms. The aim of a decision tree is to induce a classifier (a tree) from real-world example data. This tree can be used to classify unseen examples which have not been used to derive the decision tree.

A decision tree is derived from training data. An example contains values for the different attributes and what class the example belongs. In one embodiment, the training data is image data representative of an inside of a female's vessel and cardiac disease outcomes.

The following algorithm describes a decision tree derivation:

### Tree(Examples,Class,Attributes)

Create a root node
If all Examples have the same Class value, give the root this label
Else if Attributes is empty label the root according to the most
common value
Else begin
Calculate the information gain for each attribute
Select the attribute A with highest information gain and make
this the root attribute
For each possible value, v, of this attribute
Add a new branch below the root, corresponding to A=v
Let Examples(v) be those examples with A=v
If Examples(v) is empty, make the new branch a leaf node labeled
with the most common value among Examples
Else let the new branch be the tree created by

### Tree(Examples(v),Class,Attributes−{A})

end

A more detailed description of the calculation of information gain is shown in the following. If the possible classes vi of the examples have probabilities P(vi) then the information content I of the actual answer is given by:

I(P(v₁), . . . , P(vₙ))=nΣi=1−P(vᵢ)log₂ P(vᵢ)

The I-value shows how much information we need in order to be able to describe the outcome of a classification for the specific dataset used. Supposing that the dataset contains p positive (e.g. group that does not have a cardiac event) and n negative (e.g. group that does have a cardiac event) examples (e.g. individuals), the information contained in a correct answer is:

I(p/p+n,n/p+n)=−p/p+n log₂ p/p+n−n/p+n log₂ n/p+n

where log₂ is the logarithm using base two. By testing single attributes the amount of information needed to make a correct classification can be reduced. The remainder for a specific attribute A (e.g. a image) shows how much the information that is needed can be reduced.

Remainder(A)=vΣi=1pᵢ+nᵢ/p+nI(pᵢ/pi+nᵢ,nᵢ/pᵢ+nᵢ)

“v” is the number of unique attribute values for attribute A in a certain dataset, “i” is a certain attribute value, “p,” is the number of examples for attribute A where the classification is positive (e.g. group that does not have a cardiac event), “n,” is the number of examples for attribute A where the classification is negative (e.g., group that does have a cardiac event).

The information gain of a specific attribute A is calculated as the difference between the information content for the classes and the remainder of attribute A:

Gain(A)=I(p/p+n,n/p+n)−Remainder(A)

The information gain is used to evaluate how important the different attributes are for the classification (how well they split up the examples), and the attribute with the highest information.

In general there are a number of different decision tree algorithms, many of which are described in Duda, Pattern Classification, Second Edition, 2001, John Wiley & Sons, Inc. Decision tree algorithms often require consideration of feature processing, impurity measure, stopping criterion, and pruning. Specific decision tree algorithms include, cut are not limited to classification and regression trees (CART), multivariate decision trees, ID3, and C4.5.

In one approach, when an exemplary embodiment of a decision tree is used, the image data representative of an inside of a female's vessel across a training population is standardized to have mean zero and unit variance. The members of the training population are randomly divided into a training set and a test set. For example, in one embodiment, two thirds of the members of the training population are placed in the training set and one third of the members of the training population are placed in the test set. The images for a select combination of images are used to construct the decision tree. Then, the ability for the decision tree to correctly classify members in the test set is determined. In some embodiments, this computation is performed several times for a given combination of images. In each iteration of the computation, the members of the training population are randomly assigned to the training set and the test set. Then, the quality of the combination of images is taken as the average of each such iteration of the decision tree computation.

In some embodiments, the image data are used to cluster a training set. For example, consider the case in which images described in the present invention are used. Each member m of the training population will have lesion values associated with the 10 images. Such values from a member m in the training population define the vector:

| | | | | | | | | | |
|---|---|---|---|---|---|---|---|---|---|
| X₁ₘ | X₂ₘ | X₃ₘ | X₄ₘ | X₅ₘ | X₆ₘ | X₇ₘ | X₈ₘ | X₉ₘ | X₁₀ₘ |

where Xᵢₘ is the extent of the lesion of the i^{th} gene in organism m. If there are m females in the training set, selection of i images will define m vectors. Note that the methods of the present invention do not require that each the value of every single image used in the vectors be represented in every single vector m. In other words, data from a subject in which one of the images is not found can still be used for clustering. In such instances, the missing image value is assigned either a “zero” or some other normalized value. In some embodiments, prior to clustering, the image values are normalized to have a mean value of zero and unit variance.

Those members of the training population that exhibit similar images across the training group will tend to cluster together. A particular combination of images of the present invention is considered to be a good classifier in this aspect of the invention when the vectors cluster into the groups found in the training population. For instance, if the training population includes patients with good or poor cardiac disease outcomes, a clustering classifier will cluster the population into two groups, with each group uniquely representing either good or poor prognosis.

Clustering is described on pages 211-256 of Duda and Hart, Pattern Classification and Scene Analysis, 1973, John Wiley & Sons, Inc., New York. As described in Section 6.7 of Duda, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda, clustering does not require the use of a distance metric. For example, a nonmetric similarity function s(x, x′) can be used to compare two vectors x and x′. Conventionally, s(x, x′) is a symmetric function whose value is large when x and x′ are somehow “similar”. An example of a nonmetric similarity function s(x, x′) is provided on page 216 of Duda.

Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda. Criterion functions are discussed in Section 6.8 of Duda.

More recently, Duda et al., Pattern Classification, 2nd edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Nearest neighbor classifiers are memory-based and require no model to be fit. Given a query point x₀, the k training points x₍ᵣ₎, r, . . . , k closest in distance to x₀ are identified and then the point x₀ is classified using the k nearest neighbors. Ties can be broken at random. In some embodiments, Euclidean distance in feature space is used to determine distance as:

d₍ᵢ₎=∥x₍ᵢ₎−xₒ∥.

Typically, when the nearest neighbor algorithm is used, the image data used to compute the linear discriminant is standardized to have mean zero and variance 1. In the present invention, the members of the training population are randomly divided into a training set and a test set. For example, in one embodiment, two thirds of the members of the training population are placed in the training set and one third of the members of the training population are placed in the test set. Profiles represent the feature space into which members of the test set are plotted. Next, the ability of the training set to correctly characterize the members of the test set is computed. In some embodiments, nearest neighbor computation is performed several times for a given combination of images. In each iteration of the computation, the members of the training population are randomly assigned to the training set and the test set. Then, the quality of the combination of images is taken as the average of each such iteration of the nearest neighbor computation.

The nearest neighbor rule can be refined to deal with issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York.

The pattern classification and statistical techniques described above are merely examples of the types of models that can be used to construct a model for classification. It is to be understood that any statistical method can be used in accordance with the invention. Moreover, combinations of these described above also can be used. Further detail on other statistical methods and their implementation are described in U.S. patent application Ser. No. 11/134,688, incorporated by reference herein in its entirety

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, the reference set of data may be stored at a remote location and the computer communicates across a network to access the reference set to compare data derived from the female subject to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

In an exemplary embodiment shown in FIG. 1, system 200 can include a computer 249 (e.g., laptop, desktop, or tablet). The computer 249 may be configured to communicate across a network 209. Computer 249 includes one or more processor 259 and memory 263 as well as an input/output mechanism 254. Where methods of the invention employ a client/server architecture, an steps of methods of the invention may be performed using server 213, which includes one or more of processor 221 and memory 229, capable of obtaining data, instructions, etc., or providing results via interface module 225 or providing results as a file 217. Server 213 may be engaged over network 209 through computer 249 or terminal 267, or server 213 may be directly connected to terminal 267, including one or more processor 275 and memory 279, as well as input/output mechanism 271.

System 200 or machines according to the invention may further include, for any of I/O 249, 237, or 271 a video display unit (e.g., a liquid crystal display (LCD) or a cathode ray tube (CRT)). Computer systems or machines according to the invention can also include an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Memory 263, 279, or 229 according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

Exemplary step-by-step methods are described schematically in FIG. 2. It will be understood that of the methods described in FIG. 1, as well as any portion of the systems and methods disclosed herein, can be implemented by computer, including the devices described above. Image data is collected from the female subject regarding the inside of a vessel 301. This data is then inputted into the central processing unit (CPU) of a computer 302. The CPU is coupled to a storage or memory for storing instructions for implementing methods of the present invention. The instructions, when executed by the CPU, cause the CPU to provide a probability of the female subject having a cardiac event. The CPU provides this determination by inputting the subject data into an algorithm trained on a reference set of data from a plurality of women for whom image data and cardiac disease is known 303. The reference set of data may be stored locally within the computer, such as within the computer memory. Alternatively, the reference set may be stored in a location that is remote from the computer, such as a server. In this instance, the computer communicates across a network to access the reference set of data. The CPU then provides a probability the female subject having a cardiac event based on the data entered into the algorithm.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A system for determining a probability of a female subject having a cardiac event, the system comprising:
a central processing unit (CPU); and
storage coupled to said CPU for storing instructions that when executed by the CPU cause the CPU to:
accept as input, data representative of an inside of a vessel of a female subject; and
provide a probability of the subject having a cardiac event as a result of running an algorithm on said input data, the algorithm having been trained on a reference set of data from a plurality of women for whom image data and cardiac disease outcome are known.

2. The system of claim 1, wherein the data are obtained from an imaging device that images within a vessel.

3. The system according to claim 1, wherein the imaging device is selected from the group consisting of: intravascular ultrasound device and an optical coherence tomography device.

4. The system according to claim 2, wherein the data is combined with data obtained from an external imaging device that images from outside of the subject.

5. The system according to claim 1, wherein the reference set of data is selected from the group consisting of imaging data from inside a vessel, histology data, and a combination thereof.

6. The system according to claim 5, wherein the reference set of data further comprises characteristics that impact cardiovascular health.

7. The system according to claim 6, wherein the characteristics are selected from the group consisting of: age, weight, level of physical activity, smoking, and a combination thereof.

8. The system of claim 1, wherein the algorithm is stored at a remote location and the CPU communicates across a network to access said algorithm.

9. The system of claim 1, wherein the algorithm is stored locally within the CPU and the CPU accesses the algorithm within the CPU.

10. A method for determining a probability of a female subject having a cardiac event, the method comprising:
accepting as input, data representative of an inside of a vessel of a female subject; and
providing a probability of the subject having a cardiac event as a result of running an algorithm on said input data, the algorithm having been trained on a reference set of data from a plurality of women for whom image data and cardiac disease outcome are known.

11. The method of claim 10, wherein the data are obtained from an imaging device that images within a vessel.

12. The method according to claim 10, wherein the imaging device is selected from the group consisting of: intravascular ultrasound device and an optical coherence tomography device.

13. The method according to claim 11, wherein the data is combined with data obtained from an external imaging device that images from outside of the subject.

14. The method according to claim 10, wherein the reference set of data is selected from the group consisting of imaging data from inside a vessel, histology data, and a combination thereof.

15. The method according to claim 14, wherein the reference set of data further comprises characteristics that impact cardiovascular health.

16. The method according to claim 15, wherein the characteristics are selected from the group consisting of: age, weight, level of physical activity, smoking, and a combination thereof.
